(19) United States
(12) Patent Application Publication
(10) Pub. No.: US 2023/0200288 A1
(43) Pub. Date: Jun. 29, 2023

(54) METHOD FOR AN "ON-THE-FLY" TREATMENT OF AN AGRICULTURAL FIELD USING A SOIL SENSOR

(71) Applicant: BASF Agro Trademarks GmbH, Ludwigshafen am Rhein (DE)

(72) Inventors: Ole JANSSEN, Köln (DE); Nicolas WERNER, Köln (DE); Christian KERKHOFF, Köln (DE)

(21) Appl. No.: 17/927,286

(22) PCT Filed: May 28, 2021
(86) PCT No.: PCT/EP2021/064398

§ 371 Date: Nov. 22, 2022

(30) Foreign Application Priority Data
May 29, 2020 (EP) 20177478.3

(51) Int. Cl. A01B 79/00; G01N 33/24; G01N 21/3563

(52) U.S. Cl. CPC A01B 79/005; G01N 33/24; G01N 21/3563; G01N 2033/245; A01C 21/007

(57) ABSTRACT

The present invention relates to a method for treatment of an agricultural field, the method comprising the steps: 1) receiving (S10) a parametrization (10) for controlling a treatment device (200) by the treatment device (200) from a field manager system (100); 2) receiving (S20) from at least one soil sensor (400) real-time soil information on the real-world situation of the geographical location G1 in the agricultural field; 3) processing (S30) the real-time soil information to generate processed information (30), 4) determining (S40) a control signal (50) for controlling a treatment arrangement (270) of the treatment device (200) based on the received parametrization (10) and the processed information (30), 5) executing (S50) a treatment on the geographical location G2 in the agricultural field, wherein the treatment is executed based on the control signal (50) real-time after receiving the real-time soil information in such a way that the distance between location G1 and location G2 does not exceed 100 meters.

The present invention relates to a method and a treatment device for the treatment of an agricultural field.

### BACKGROUND OF THE INVENTION

The general background of this invention is the treatment of an agricultural field. This treatment comprises seeding—i.e. spreading of the seeds of the crops to be cultivated—, the treatment of the actual crops to be cultivated, the treatment of weed in the agricultural field, the treatment of the insects or other animal pests in the agricultural field, the treatment of pathogens in the agricultural field, the irrigation and the fertilization of the agricultural field.

Agricultural machines or automated treatment devices, like smart sprayers, treat the weed, the insects and/or the pathogens in the agricultural field based on ecological and economical rules.

Modern agricultural machines get equipped with more and more sensors, measuring or determining different parameters relevant for the treatment of an agricultural field. Important parameters in this context are the soil condition and other soil-related parameters. The soil condition characterized for example by moisture and nutrient content is key for plant growth and health and has a crucial impact on the treatment parameters (e.g. treatment type and fertilizer dosage). In the state of the art, the soil condition is either measured via a limited number of point measurements where data collection and analytics is time-consuming and costly, or the soil condition is determined via estimations based on physical models, e.g. based on remote sensing information. Both methods lack the required accuracy for digital farming applications. Other methods in the prior art solutions don't perform real-time decision making, thus require multiple field visits which are time-consuming.

### SUMMARY OF THE INVENTION

It would be advantageous to have an improved method for the treatment of an agricultural field which provides a high-quality real-time measurement and determination of the soil condition and/or other soil-related parameters and at the same time a real-time decision and/or execution regarding the treatment of an agricultural field. Furthermore, it would be advantageous to have an improved method which allows for a precise, zone-specific and soil-parameter-dependent treatment of an agricultural field. Furthermore, it would be advantageous to have a cost-efficient method which allows for a precise, zone-specific and soil-parameter-dependent where there is no need to drag sensors through the soil. Furthermore, it would be advantageous to have a cost-efficient method which allows for a precise, zone-specific and soil-parameter-dependent where there is no need to drag sensors through the soil where there is no need to mechanically attach a soil sensor to the treatment device.

The object of the present invention is solved with the subject matter of the independent claims, wherein further embodiments are incorporated in the dependent claims. It should be noted that the following described aspects and examples of the invention apply for the method as well as for the treatment device.

In view of the above object(s) of the present invention, the present invention relates to a method for treatment of an agricultural field (300), the method comprising the steps:
1) receiving (S10) a parametrization (10) for controlling a treatment device (200) by the treatment device (200) from a field manager system (100);
2) receiving (S20)—from at least one soil sensor (400)—real-time soil information on the real-world situation of the geographical location G1 in the agricultural field;
3) processing (S30) the real-time soil information to generate processed information (30),
4) determining (S40) a control signal (50) for controlling a treatment arrangement (270) of the treatment device (200) based on the received parametrization (10) and the processed information (30),
5) executing (S50) a treatment on the geographical location G2 in the agricultural field,

wherein the treatment is executed based on the control signal (50) real-time after receiving the real-time soil information in such a way that the distance between location G1 and location G2 does not exceed 100 meters.

In view of the above object(s) of the present invention, the present invention relates to a method for treatment of an agricultural field (300), the method comprising the steps:
1) receiving (S10) a parametrization (10) for controlling a treatment device (200) by the treatment device (200) from a field manager system (100), wherein the parametrization (10) is dependent on offline field data (Doff) relating to expected conditions on the agricultural field (300);
2) receiving (S20)—from at least one soil sensor (400)—real-time soil information on the real-world situation of the geographical location G1 in the agricultural field;
3) processing (S30) the real-time soil information to generate processed information (30),
4) determining (S40) a control signal (50) for controlling a treatment arrangement (270) of the treatment device (200) based on the received parametrization (10) and the processed information (30),
5) executing (S50) a treatment on the geographical location G2 in the agricultural field,

wherein the treatment is executed based on the control signal (50) real-time after receiving the real-time soil information in such a way that the distance between location G1 and location G2 does not exceed 100 meters.

In view of the above object(s) of the present invention, the present invention also relates to a treatment device (200) for treatment of an agricultural field (300), comprising:
a soil sensor (400),
a processing unit (500) being adapted for processing the real-time soil information on the real-world situation of the geographical location G1 in the agricultural field (300) as received from the soil sensor (400) and generating processed information (30),
a parametrization interface (250) being adapted for receiving a parametrization (10) from a field manager system (100),
a treatment arrangement (270) being adapted for treating the agricultural field (300) dependent on the control signal (50) and being adapted for executing a treatment on the geographical location G2 in the agricultural field (300) real-time after receiving the real-time soil information in such a way that the distance between location G1 and location G2 does not exceed 100 meters;
a treatment control unit (210) being adapted for determining a control signal (50) for controlling a treatment arrangement (270) based on the parametrization (10) which it receives from the parametrization interface (250) and based on the processed information (30).

In this context, “comprising” (a soil sensor, a processing unit, a parametrization interface, a treatment arrangement, a treatment control unit) in relationship to the treatment device (200) means, that the treatment device (200) is communicatively coupled to a soil sensor, a processing unit, a parametrization interface, a treatment arrangement, or a treatment control unit. It is not required that the soil sensor, processing unit, parametrization interface, treatment arrangement, or treatment control unit is mechanically attached or mechanically part of the treatment device (200). The term “communicatively coupled” means that two parts or devices or hardware pieces are coupled in a way that they can communicate with each other, for example via mobile internet (2G/3G/4G/5G/6G) connections or wireless internet connections (e.g. WiFi).

The term “treatment” or “treatment of an agricultural field”, as used herein, preferably comprises:
protecting a crop or plant, which is cultivated or is to be cultivated on an agricultural field, via destroying a weed that is not cultivated and may be harmful for the crop, in particular with a herbicide, via controlling or killing insects or animal pests on the crop and/or the weed, in particular with an insecticide, and via controlling or destroying any pathogens and/or plant diseases on the crop, in particular with a fungicide, and/or
regulating the growth of crop or plants on an agricultural field, in particular with a plant growth regulator, and/or
seeding, i.e. spreading or planting the seeds or seedlings of the crops or plants to be cultivated on an agricultural field, and/or
providing fertilizers or nutrients to the crop or plant which is cultivated or is to be cultivated on an agricultural field, and/or
irrigation of an agricultural field.

The term “offline field data” as used herein refers to any data generated, collected, aggregated or processed before determination of the parametrization. The offline field data may be collected externally from the treatment device. The offline field data may be data collected before the treatment device is being used. The offline field data may be data collected before the treatment is conducted in the agricultural field based on the received parametrization. Offline field data for instance includes weather data associated with expected weather conditions at the time of treatment, expected soil data associated with expected soil conditions, e.g. nutrient content, soil moisture, and/or soil composition, at the time of treatment, growth stage data associated with the growth stage of e.g. a weed or crop at the time of treatment, and/or disease data associated with the disease stage of a crop at the time of treatment. More preferably, the offline field data may also comprise static data (i.e. data which are not dynamically changing) of the agricultural machine on which the treatment device is mounted. For example, static machine data are machine specification data, machine dimensions (e.g. machine length, breadth, and height), static machine properties data etc. of the agricultural machine on which the treatment device is mounted.

The term “spatially resolved” as used herein refers to any information on a sub-field scale. Such resolution may be associated with more than one location coordinate on the agricultural field or with a spatial grid of the agricultural field having grid elements on a sub-field scale. In particular, the information on the agricultural field may be associated with more than one location or grid element on the agricultural field. Such spatial resolution on sub-field scale allows for more tailored and targeted treatment of the agricultural field.

The term “condition on the agricultural field” relates to any condition of the agricultural field or environmental condition in the agricultural field, which has impact on the treatment of the agricultural field. Such condition may be associated with the soil or weather condition. The soil condition may be specified by soil data relating to a current or expected condition of the soil. The weather condition may be associated with weather data relating to a current or expected condition of the weather. The growth condition may be associated with the growth stage of e.g. a crop or weed. The disease condition may be associated with the disease data relating to a current or expected condition of the disease.

The term “treatment device”, as used herein or also called control technology, may comprise chemical control technology, or seed control technology, or irrigation control technology. Chemical control technology preferably comprises at least one means for application of treatment products, particularly crop protection products like insecticides and/or herbicides and/or fungicides. Such means may include a treatment arrangement of one or more spray guns or spray nozzles arranged on an agricultural machine, drone or robot for maneuvering through the agricultural field. Seed control technology preferably comprises at least one means for application of seeds, including equipment for seed broadcasting, dibbing, seed dropping behind the plough, drilling, hill dropping, check rowing and transplanting. For example, seed control technology may include a regular drill planter, in which for instance the seeds are picked from the hopper by a specific circular-shaped plate and released in the shank to be delivered through gravity to the bottom of the furrow. The treatment arrangement is a part or section of the treatment device which actually executes the treatment, for example the treatment arrangement can be a nozzle arrangement (in case of spraying device) or an arrangement of circular-shaped plates (in case of seeding device). The treatment arrangement is preferably mechanically attached to the treatment device. The treatment arrangement may also be not mechanically attached to the treatment device. The treatment arrangement is preferably communicatively coupled to the treatment device.

The term “parametrization”, as used herein, relates to a set of parameters provided to a treatment device for controlling the treatment device treating the agricultural field. The parametrization for controlling the treatment device may be at least partially spatially resolved for the agricultural field or at least partially location specific. Such spatial resolution or location specificity may be based on spatially resolved offline field data. Spatially resolved offline data may include spatially resolved historic or modelling data of the agricultural field. Alternatively or additionally, spatially resolved offline data may be based on remote sensing data for the agricultural field or observation data detected at limited number of locations in the agricultural field. Such observation data may include images detected in certain locations of the agricultural field e.g. via a mobile device, and optional outcomes derived via image analysis.

The parametrization may relate to a configuration file for the treatment device, which may be stored in memory of the treatment device and accessed by the control unit of the treatment device. In other words, the parametrization may be a logic e.g. a decision tree with one or more layers, which is used to determine a control signal for controlling the treatment device dependent on measurable input variables e.g. images taken and/or online field data. The parametrization may include one layer relating to an on/off decision and optionally a second layer relating to a composition of the treatment product expected to be used and further optionally a third layer relating to a dosage of the treatment product expected to be used. Out of these layers of parametrization the on/off decision, the composition of the treatment product and/or the dosage of the treatment product may spatially resolved or location specific for the agricultural field. In such way a situational, real-time decision on treatment is based on the real-time processed information and/or online field data collected while the treatment device passages the agricultural field. Providing a parametrization prior to the execution of treatment reduces the computing time and at the same time enables reliable determination of control signals for treatment. The parametrization or configuration file may include location specific parameters provided to the treatment device, which may be used to determine the control signal.

In one layer the parametrization for on/off decisions may include thresholds relating to a parameter(s) derived from the processed information. Such parameters may be derived from the processed information (30) and decisive for the treatment decision. In a preferred embodiment the parameters derived from the processed information relates to the soil condition. Further parameters may be derived from online field data decisive for the treatment decision. Is the derived parameter e.g. below the threshold the decision is off or no treatment. Is the derived parameter e.g. above the threshold the decision is on or treatment. The parametrization may include a spatially resolved set of thresholds. In such way the control signal is determined based on the parametrization and the processed information (30).

Preferably, the treatment device is provided with a parametrization or configuration file, based on which the treatment device controls the treatment arrangement. In a further embodiment determination of the configuration file comprises a determination of a dosage level the treatment product is to be applied. The parametrization may include a further layer on dosage of the treatment product. Such dosage may relate to a derived parameter from the processed information (30). Further parameters may be derived from online field data. In other words, based on the configuration file the treatment device is controlled, as to which dose of the treatment product should be applied based on real-time parameters of the agricultural field, such as images taken and/or online field data. In a preferred embodiment the parametrization includes variable or incremental dosage levels depending on one or more parameter(s) derived from the processed information (30). In a further preferred embodiment, determining a dosage level is based on the processed information (30). The parametrization may include a spatially resolved set of dosage levels.

The parametrization may include a further layer on the composition of the treatment product expected to be used. In such a case the parametrization may be determined depending on an expected significant yield or quality impact on the crop, an ecological impact and/or costs of the treatment product composition. Therefore, based on the parametrization, the decision, if a field is treated or not and with which treatment product composition at which dosage level it should be treated is taken for the best possible result in regard of efficiency and/or efficacy. The parametrization may include a tank recipe for a treatment product tank system of the treatment device. In other words, the treatment product composition may signify the treatment product components provided in one or more tank(s) of the treatment device prior to conducting the treatment. Mixtures from one or more tank(s) forming the treatment product may be controlled on the fly depending on the determined composition of the treatment product. The treatment product composition may be determined based on the processed information (30). Additionally or alternatively, the parametrization may include a spatially resolved set of treatment product compositions expected to be used.

The term “dosage level”, as used herein, preferably refers to the quantity of seeds, the quantity of fertilizers, the quantity of water, and/or the quantity of crop protection product applied within a certain area, for example applied on one hectare.

In a preferred embodiment the treatment device comprises one or more spray gun(s), and/or circular plates or rotating plates (for example as part of a regular drill planter) and/or associated image capture device(s). The image capture devices may be arranged such that the images are associated with the area to be treated by the one or more spray gun(s). The image capture devices may for instance be mounted such that an image in direction of travel of the treatment device is taken covering an area that is to be treated by the respective spray gun(s). Each image may be associated with a location and as such provide a snapshot of the real time situation in the agricultural field prior to treatment. Hence the image capture devices may take images of specific locations of the agricultural field as the treatment device traverses through the agricultural field and the control signal may be adapted accordingly based on the image taken of the area to be treated. The control signal may hence be adapted to the situation captured by the image at the time of treatment in a specific location of the agricultural field.

Preferably, the control signal for controlling the treatment device may be determined based on the received parametrization, the processed information (30) and online field data. In one embodiment online field data is collected in real time in particular by the treatment device. Collecting online field data may include collecting sensor data from any sensors (including cameras) attached to the treatment device or placed in the agricultural field in particular on the fly or in real time as the treatment device passages the agricultural field. Collecting online field data may particularly include weather data collected via weather sensory placed in or in proximity to the agricultural field or attached to the treatment device and associated with a current weather condition or data.

The term “efficiency” relates to balance of the amount of treatment product applied and the amount of treatment product needed to effectively treat the crops or plants on the agricultural field. How efficiently a treatment is conducted depends on environmental factors such as weather and soil.

The term “efficacy” relates to the balance of positive and negative effects of a treatment product. In other words, efficacy relates to the optimal dose of treatment product needed to effectively treat a specific crop or plant on an agricultural field. The dose should not be so high that treatment product is wasted, which would also increase the costs and the negative impact on the environment, but is not so low that the treatment product is not effectively treated, which could lead to immunization of the crop or plant against the treatment product. Efficacy of a treatment product also depends on environmental factors such as weather and soil.

The term “treatment product”, as used herein, refers to products for treatment of an agricultural field such as water (used for irrigation), herbicides, insecticides, fungicides, plant growth regulators, nutrition products and/or mixtures thereof. The treatment product may comprise different components—including different active ingredients—such as different herbicides, different fungicides, different insecticide, different nutrition products, different nutrients, as well as further components such as safeners (particularly used in combination with herbicides), adjuvants, fertilizers, co-formulants, stabilizers and/or mixtures thereof. The term “treatment product composition” thereby relates to different active ingredient(s) contained in the treatment product, particularly, the treatment product composition is a composition comprising one, or two, or more treatment products. Thus, there are different types of e.g. herbicides, insecticides and/or fungicides, respectively based on different active ingredient(s). Since the plant to be protected by the treatment product preferably is a crop, the treatment product can be referred to as crop protection product. The treatment product composition may also comprise additional substances that are mixed to the treatment product, like for example water, in particular for diluting and/or thinning the treatment product, and/or a nutrient solution, in particular for enhancing the efficacy of the treatment product. Preferably, the nutrient solution is a nitrogen-containing solution, for example liquid urea ammonium nitrate (UAN).

The term “product” or “treatment product” is understood to be any object or material useful for the treatment. In the context of the present invention, the term “product” includes but is not limited to:
chemical products such as fungicide, herbicide, insecticide, acaricide, molluscicide, nematicide, avicide, piscicide, rodenticide, repellant, bactericide, biocide, safener, plant growth regulator, urease inhibi-tor, nitrification inhibitor, denitrification inhibitor, or any combination thereof.
biological products such as microorganisms useful as fungicide (biofungicide), herbicide (bioherbicide), insecticide (bioinsecticide), acaricide (bioacaricide), molluscicide (biomolluscicide), nematicide (bionematicide), avicide, piscicide, rodenticide, repellant, bactericide, biocide, safener, plant growth regulator, urease inhibitor, nitrification inhibitor, denitrification inhibitor, or any combination thereof,
fertilizer and nutrient,
seed and seedling,
seed treatment products (including seed treatment products used for in-furrow seed treatment) or seed coating products,
water, and
any combination thereof.

The term “insecticide”, as used herein, also encompasses nematicides, acaricides, and molluscicides.

The term “nutrition product”, as used herein, refers to any products which are beneficial for the plant nutrition and/or plant health, including but not limited to fertilizers, macronutrients and micronutrients.

The term “determine” also includes “initiate the determination” or “cause the determination”.

Including a pre-determined parametrization into the treatment device control improves the decision making and hence the efficiency of the treatment and/or the efficacy of the treatment product. In particular, the location specific image or online field data can be processed more efficiently via the pre-determined parametrization. An—at least in part spatially resolved—parametrization further improves the control of the treatment device on the fly during treatment. Thus, an improved method for the treatment of an agricultural field improving economic return of investment and improving an impact into the ecosystem is provided.

### PREFERRED EMBODIMENTS

According to a preferred embodiment of the present invention, the treatment is executed based on the control signal (50) real-time after receiving the real-time soil information in such a way that the distance between location G1 and location G2 does not exceed 100 meters, preferably does not exceed 80 meters, more preferably does not exceed 60 meters, most preferably does not exceed 50 meters, particularly preferably does not exceed 40 meters, particularly more preferably does not exceed 30 meters, particularly most preferably does not exceed 25 meters, particularly does not exceed 20 meters, for example preferably does not exceed 15 meters, for example more preferably does not exceed 10 meters, for example preferably does not exceed 8 meters, for example does not exceed 6 meters, for instance preferably does not exceed 5 meters, for instance more preferably does not exceed 4 meters, for instance preferably does not exceed 3 meters, for instance does not exceed 2 meters, in particular does not exceed 1.5 meters, in particular preferably does not exceed 1 meter. According to a preferred embodiment of the present invention, the treatment is executed based on the control signal (50) real-time after receiving the real-time soil information in such a way that the distance between location G1 and location G2 is at least 0.01 meters, preferably at least 0.05 meters, more preferably at least 0.1 meters, most preferably at least 0.3 meters, particularly preferably at least 0.5 meters, particularly more preferably at least 0.7 meters, particularly most preferably at least 0.9 meters, particularly at least 1 meter, for example preferably at least 1.2 meters, for example more preferably at least 1.4 meters, for example preferably at least 1.5 meters. In general, this distance may be dependent on the treatment device or dependent on the carrier which carries or transports the treatment device on its way over the agricultural field, such carrier may be an agricultural machine, a drone, a robot etc. Furthermore, this distance may also be dependent on the speed with which the carrier which carries or transports the treatment device traverses over the agricultural field.

According to a preferred embodiment of the present invention, the real-time soil information received by the soil sensor in step 2) comprises:
biological information such as information regarding the microbial activity of the soil, and/or
physical information such as information regarding the soil texture, soil conductivity, soil moisture, soil density, and/or soil temperature, and/or
chemical information such as information regarding the nutrient content of the soil, humus content of the soil, carbonate content of the soil, dry matter content, total carbon content, organic carbon content, boron content, phosphorus content, potassium content, nitrogen content, sulfur content, calcium content, iron content, aluminum content, chlorine content, molybdenum content, magnesium content, nickel content, copper content, zinc content, and/or Manganese content of the soil, chemical composition of the soil, soil salinity, and/or pH value of the soil.

According to a preferred embodiment of the present invention, the real-time soil information received by the soil sensor in step 2) comprises biological information such as information regarding the microbial activity of the soil.

According to a preferred embodiment of the present invention, the real-time soil information received by the soil sensor in step 2) comprises physical information such as information regarding the soil texture, soil conductivity, soil moisture, soil density, and/or soil temperature.

According to a preferred embodiment of the present invention, the real-time soil information received by the soil sensor in step 2) comprises chemical information such as information regarding the nutrient content of the soil, humus content of the soil, carbonate content of the soil, dry matter content, total carbon content, organic carbon content, boron content, phosphorus content, potassium content, nitrogen content, sulfur content, calcium content, iron content, aluminum content, chlorine content, molybdenum content, magnesium content, nickel content, copper content, zinc content, and/or Manganese content of the soil, chemical composition of the soil, soil salinity, and/or pH value of the soil.

According to a preferred embodiment of the present invention, the control signal (50) is determined within 100 seconds, more preferably within 50 seconds, most preferably within 30 seconds, particularly preferably within 20 seconds, particularly more preferably within 15 seconds, particularly most preferably within 10 seconds, particularly within 7 seconds, for example preferably within 5 seconds, for example more preferably within 3 seconds, for example most preferably within 2 seconds, for example within 1 second, for instance within 500 ms, for instance preferably within 100 ms after receiving the real-time soil information in step 2).

According to a preferred embodiment of the present invention, executing (S50) a treatment on the geographical location G2 in the agricultural field (step 5) takes place within 100 seconds, more preferably within 50 seconds, most preferably within 30 seconds, particularly preferably within 20 seconds, particularly more preferably within 15 seconds, particularly most preferably within 10 seconds, particularly within 7 seconds, for example preferably within 5 seconds, for example more preferably within 3 seconds, for example most preferably within 2 seconds, for example within 1 second, for instance within 500 ms, for instance preferably within 100 ms after receiving the real-time soil information in step 2).

According to a preferred embodiment of the present invention (Embodiment 2), a method according to the present invention comprises the additional steps:
receiving the offline field data (Doff) by the field manager system (100);
determining the parametrization (10) of the treatment device (200) dependent on the offline field data (Doff) and determining a dosage level (40) or determining at least one treatment product type (41); and
providing the determined parametrization (10) and the determined dosage level (40) or the determined treatment product type (41) to the treatment device (200).

According to a preferred embodiment of the present invention (Embodiment 3), the physical distance between the soil sensor and the soil is less than 400 km, more preferably less than 100 km, most preferably less than 10 km, particularly preferably less than 1 km, particularly more preferably less than 300 m, particularly more preferably less than 100 m, especially preferably less than 30 m, especially more preferably less than 10 m, especially most preferably less than 3 m, especially less than 1 m, for instance preferably less than 60 cm, for instance more preferably less than 30 cm, for instance most preferably less than 10 cm, for instance less than 3 cm, for example preferably less than 1 cm, for example more preferably less than 5 mm, for example less than 1 mm at the time of obtaining real-time soil information on the real-world situation in the agricultural field. According to a preferred embodiment of the present invention, the soil sensor is a remote sensor—e.g. a remote sensor being part of or being mounted on an aerial vehicle, an unmanned aerial vehicle, or a satellite—, wherein more preferably the distance between the soil sensor and the soil is at least 1 m at the time of obtaining real-time soil information on the real-world situation in the agricultural field. According to a preferred embodiment of the present invention, the soil sensor is a proximal sensor, wherein more preferably the distance between the soil sensor and the soil is not more than 1 m at the time of obtaining realtime soil information on the real-world situation in the agricultural field.

According to a preferred embodiment of the present invention (Embodiment 4), the soil sensor is a non-optical spectrometer, an optical spectrometer, an infrared spectrometer, an electric conductivity sensor, a magnetic susceptibility (EM) sensor, a gamma-ray sensor, a Lidar sensor, a near-infrared sensor, or a photoconductive-layer-containing optical sensor. According to another preferred embodiment of the present invention, the soil sensor is an infrared spectrometer, or a photoconductive-layer-containing optical sensor.

The photoconductive-layer-containing optical sensor is preferably a sensor described in the patent application WO2018/019921. The photoconductive-layer-containing optical sensor is more preferably an optical sensor, comprising a layer of at least one photoconductive material, at least two individual electrical contacts contacting the layer of the photoconductive material, and a cover layer deposited on the photoconductive material, wherein the cover layer is an amorphous layer comprising at least one metal-containing compound. The photoconductive-layer-containing optical sensor is most preferably an optical sensor, comprising a layer of at least one photoconductive material, at least two individual electrical contacts contacting the layer of the photoconductive material, and a cover layer deposited on the photoconductive material, wherein the cover layer is an amorphous layer comprising at least one metal-containing compound, wherein the at least one metal-containing compound comprises a metal selected from the group consisting of Al, Ti, Ta, Mn, Mo, Zr, Hf and W. The photoconductive-layer-containing optical sensor is even more preferably an optical sensor, comprising a layer of at least one photoconductive material, at least two individual electrical contacts contacting the layer of the photoconductive material, and a cover layer deposited on the photoconductive material, wherein the cover layer is an amorphous layer comprising at least one metal-containing compound, wherein the photoconductive material comprises at least one chalcogenide, wherein the chalcogenide is selected from the group consisting of lead sulfide (PbS), copper indium sulfide (CIS), copper indium gallium selenide (CIGS), copper zinc tin sulfide (CZTS), lead selenide (PbSe), copper zinc tin selenide (CZTSe), cadmium telluride (CdTe), mercury cadmium telluride (HgCdTe), mercury zinc telluride (HgZnTe), lead sulfoselenide (PbSSe), copper-zinc-tin sulfur-selenium chalcogenide (CZTSSe), and a solid solution and/or a doped variant thereof. The photoconductive-layer-containing optical sensor has preferably a compact design, a high wavelength resolution (e.g. below 50 nm, e.g. preferably below 30 nm, e.g. preferably below 20 nm), and the wavelength range in which it is operating is preferably between 1 μm and 3 μm, more preferably between 1.2 and 2.6 μm.

According to a preferred embodiment of the present invention (Embodiment 5), the soil sensor is an infrared spectrometer optionally supplemented by one of the sensors selected from non-optical spectrometer, optical spectrometer, electric conductivity sensor, gamma-ray sensor, magnetic susceptibility (EM) sensor, and/or optionally supplemented by a camera.

According to a preferred embodiment of the present invention (Embodiment 6), the soil sensor is a photoconductive-layer-containing optical sensor optionally supplemented by one of the sensors selected from non-optical spectrometer, optical spectrometer, electric conductivity sensor, gamma-ray sensor, magnetic susceptibility (EM) sensor, and/or optionally supplemented by a camera.

According to a preferred embodiment of the present invention (Embodiment 7), the soil sensor (400) is mechanically attached to the treatment device (200). Preferably, the soil sensor is attached to any mechanical part of the treatment device or any mechanical part of the agricultural machine on which the treatment device is mounted. More preferably, the soil sensor (400) is attached to a mechanical part of the treatment device or any mechanical part of the agricultural machine on which the treatment device is mounted, wherein the mechanical part is dragged through the soil, or has direct contact with the soil.

According to a preferred embodiment of the present invention (Embodiment 8), the soil sensor (400) is not mechanically attached to the treatment device (200), and is directly or indirectly communicatively coupled to the treatment device (200), and more preferably, such a soil sensor (400) is:
a soil sensor (404) which is not mechanically attached to the treatment device (200), and fixed on the specific location of the agricultural field, for example soil moisture sensor fixed in the soil, or
a soil sensor (406) which is not mechanically attached to the treatment device (200), and mobile or movable depending on the movement of the treatment device, for example a soil sensor which is part of or mounted on an aerial vehicle, an unmanned aerial vehicle, a drone, or a robot,
a soil sensor (408) which is not mechanically attached to the treatment device (200), and mobile or movable independent from on the movement of the treatment device, for example a soil sensor is part of or mounted on an aerial vehicle, an unmanned aerial vehicle, or a satellite.

According to a preferred embodiment of the present invention (Embodiment 9), the treatment device (200) is designed as a smart “seed applicator”, wherein the treatment arrangement (270) is a seeding arrangement.

According to a preferred embodiment of the present invention (Embodiment 10), the treatment device (200) is designed as a smart “fertilizer applicator”, wherein the treatment arrangement (270) is a fertilizing arrangement.

According to a preferred embodiment of the present invention (Embodiment 11), wherein the treatment device (200) is designed as a smart sprayer, wherein the treatment arrangement (270) is a nozzle arrangement.

According to a preferred embodiment of the present invention (Embodiment 12), wherein the treatment device (200) is designed as a smart “irrigation applicator”, wherein the treatment arrangement (270) is an irrigation arrangement.

According to a preferred embodiment of the present invention (Embodiment 13), the offline field data (Doff) includes at least one type of the following data types:
local yield expectation data,
resistance data relating to a likelihood of resistance of the plantation against a treat-ment product,
expected weather condition data,
expected plantation growth data,
zone information data relating to different zones of the agricultural field,
expected soil data,
legal restriction data, particularly regulatory data,
weather data associated with expected weather conditions at the time of treatment,
expected soil data associated with expected soil conditions, nutrient content or soil moisture at the time of treatment,
soil composition at the time of treatment,
growth stage data associated with the growth stage of a weed or crop at the time of treatment,
disease data associated with the disease stage of a crop at the time of treatment, and/or
further data having an impact on the treatment including data relating to crop type, crop species, seed variety, geographic location, crop demand, soil water availability, soil depth, leaching potential, run-off risk, erosion risk.

According to a preferred embodiment of the present invention (Embodiment 14), the method of the present invention comprises the steps:
receiving online field data (Don) by the treatment device (200) relating to current conditions on the agricultural field (300); and
determining the control signal (50) dependent on the determined parametrization (10), the processed information (30), and the determined online field data (Don).

According to a preferred embodiment of the present invention, the method of the present invention comprises the steps:
receiving online field data (Don) by the treatment device (200) relating to current conditions on the agricultural field (300); and
determining the control signal (50) dependent on the determined parametrization (10), the chosen dosage level (40) or the chosen treatment product type (41), the processed information (30) and the determined online field data (Don).

According to a preferred embodiment of the present invention (Embodiment 15), the online field data (Don) relates to current machine data, current weather condition data, and current plantation growth data. Current machine data are any dynamically changing data of the agricultural machine on which the treatment device is mounted. For example, current machine data are current speed data, current geographical position data, current driving direction data, current elevation data etc. of the agricultural machine on which the treatment device is mounted.

According to a preferred embodiment of the present invention (Embodiment 17), the method of the present invention comprises the step:

adjusting the parametrization (10) and/or the dosage level (40) or the at least one treatment product type (41) using a machine learning algorithm.

According to a preferred embodiment of the present invention (Embodiment 18), the method of the present invention comprises the step:

processing (S30) the real-time soil information to generate processed information (30) using a machine learning algorithm.

Regarding the machine learning algorithm for the processing (S30) of the real-time soil information to generate processed information (30), historic datasets are used, for example historic datasets of soil reflectance data and nitrogen content data are used as input, and a “soil reflectance versus nitrogen content” correlation curve or graph is outputted.

According to a preferred embodiment of the present invention, processing (S30) of the real-time soil information includes
the analysis of the spectrum outputted by the soil sensor,
calibrating and/or adjusting the raw data outputted by the soil sensor,
deriving and/or outputting of specific soil parameter data, or
data processing using an agronomic model including a decision logic. The decision logic for example comprises a logic according to which a certain amount of nitrogen-containing fertilizer will be applied in step 5 in case the nitrogen content of the soil is below a predefined threshold value.

According to a preferred embodiment of the present invention, processing (S30) of the real-time soil information includes translating proxy parameters—i.e. those parameters not directly useful for determining a control signal (50)—, such as the reflectance of soil or soil parts which is measured by soil sensors, to useful parameters, such as the amount of nitrogen in the soil which can be obtained from the reflectance data via data translation.

According to a preferred embodiment of the present invention, processing (S30) of the real-time soil information (20) may also be dependent on the offline field data and online field data, particularly for validating, calibrating and adjusting the processed information (30). According to a preferred embodiment of the present invention, processing (S30) of the real-time soil information may also be dependent on the online field data, particularly for validating, calibrating and adjusting the processed information (30). For example, in case the real-time soil-information is soil moisture, and in case the online field data comprises the information about precipitation in the last days, during the processing (S30) step, the real-time soil-information about soil moisture can be validated, calibrated or adjusted according to the online field data. In this exemplary case, if the real-time soil-information shows that the soil moisture is around zero (e.g. because the soil sensor is not working properly), and the online field data shows that there was very heavy rainfall in the last days, then the during processing (S30) step, the soil moisture maybe adjusted to a higher value, e.g. according to a model which correlates precipitation with soil moisture.

The machine-learning algorithm may comprise decision trees, naive bayes classifications, nearest neighbors, neural networks, convolutional neural networks (CNNs) or recurrent neural networks, generative adversarial networks, support vector machines, linear regression, logistic regression, random forest, ResNet and/or gradient boosting algorithms.

According to a preferred embodiment of the present invention (Embodiment 19), determining a parametrization (10) comprises determining a tank recipe for a treatment product tank of the treatment device (200).

According to a preferred embodiment of the present invention (Embodiment 21), the treatment device (200) comprises:

an online field data interface (240) being adapted for receiving online field data (Don) relating to current conditions on the agricultural field (300); wherein

the treatment control unit (210) is adapted for determining a control signal (50) for control-ling a treatment arrangement (270) dependent on the received parametrization (10) and the processed information (30) and/or the online field data (Don).

According to a preferred embodiment of the present invention (Embodiment 22), the treatment device (200) is designed as a smart seed applicator, wherein the treatment arrangement (270) is a seeding arrangement.

According to a preferred embodiment of the present invention (Embodiment 23), the treatment device (200) is designed as a smart sprayer, wherein the treatment arrangement (270) is a nozzle arrangement.

The following reference list is used in the present application.

### REFERENCE LIST

10 parametrization
20 real-time soil information
30 processed information
40 dosage level
41 treatment product type
50 control signal
100 field manager system
200 treatment device
210 treatment control unit
240 online field data interface
250 parametrization interface
270 treatment arrangement
300 agricultural field
400 soil sensor
402 soil sensor mechanically attached to the treatment device
404 soil sensor not mechanically attached to the treatment device but communicatively coupled to the treatment device, and fixed on the specific location of the agricultural field,
406 soil sensor not mechanically attached to the treatment device but communicatively coupled to the treatment device, and mobile or movable depending on the movement of the treatment device
408 soil sensor not mechanically attached to the treatment device but communicatively coupled to the treatment device, and mobile or movable independent from on the movement of the treatment device
500 processing unit
G1 Geographical location from which real-time soil information on the real-world situation is received from at least one soil sensor
G2 Geographical location where the treatment is executed

The above mentioned and other aspects of the invention will be apparent from and elucidated further with reference to the embodiments described by way of examples in the following description and with reference to the accompanying drawings, in which

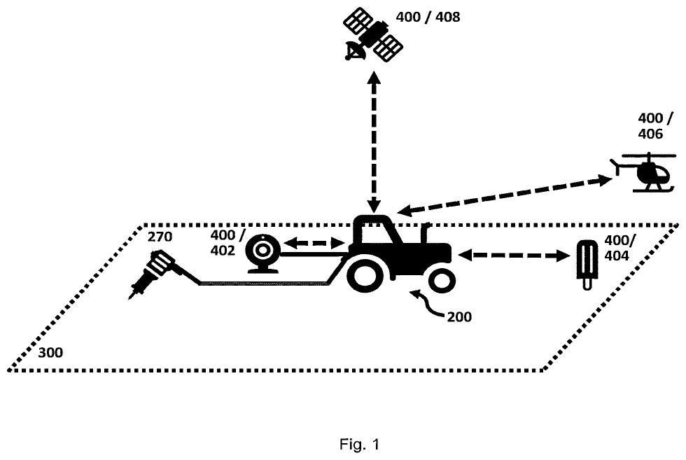

FIG. 1 illustrates one example of a treatment device comprising one of the four different types of soil sensors;

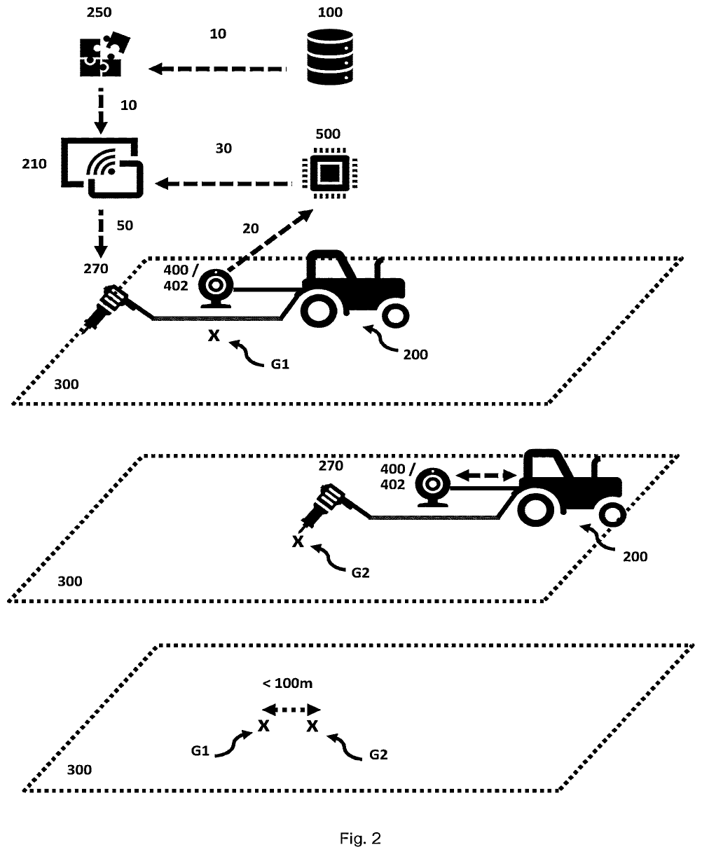

FIG. 2 illustrates one example of an executed treatment of the treatment device comprising a mechanically attached soil sensor;

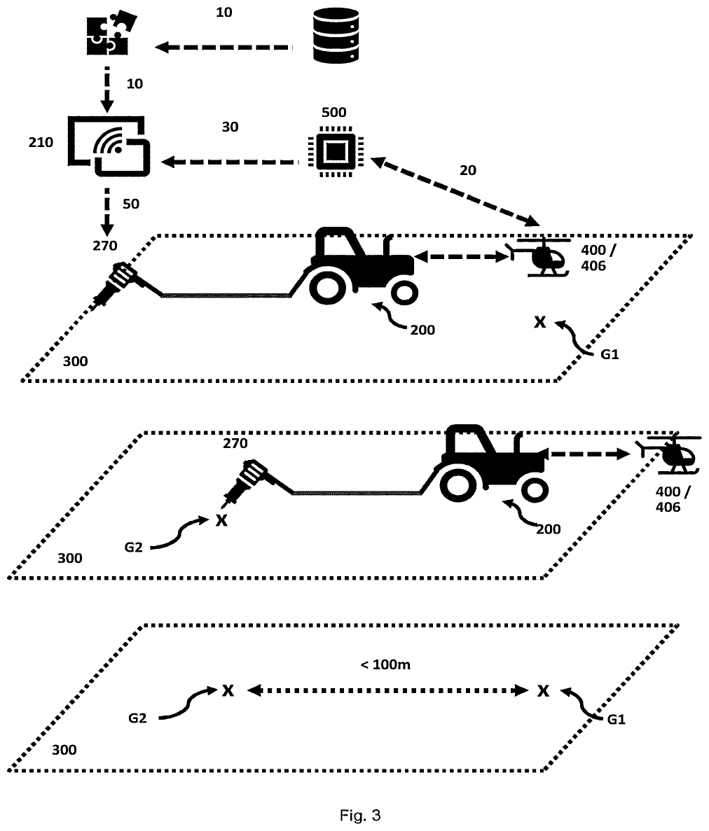

FIG. 3 illustrates one example of an executed treatment of the treatment device comprising a mobile soil sensor not mechanically attached but communicatively coupled to the treatment device and moving depending on the movement of the treatment device;

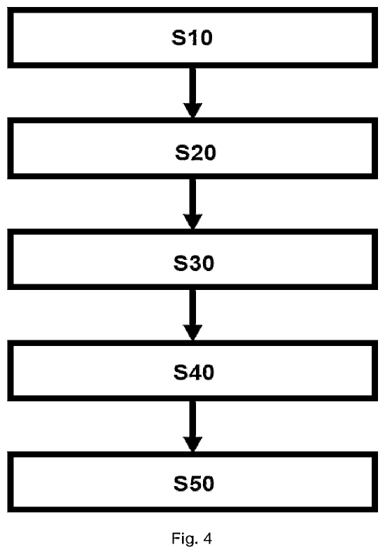

FIG. 4 illustrates a flow diagram showing the operation of the method of the present invention.

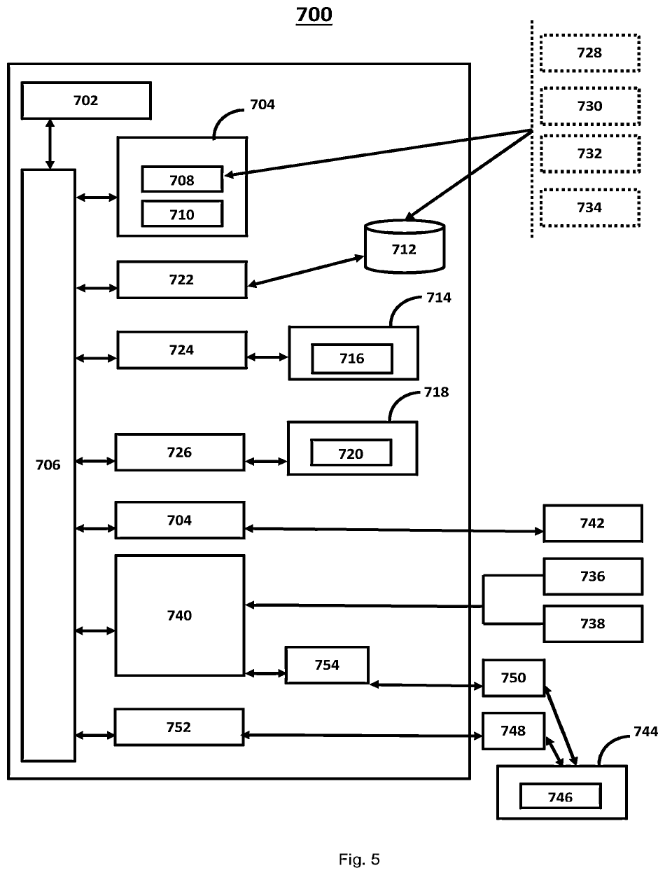

FIG. 5 illustrates an embodiment of an exemplary computing architecture 700 suitable for implementing various embodiments as previously described.

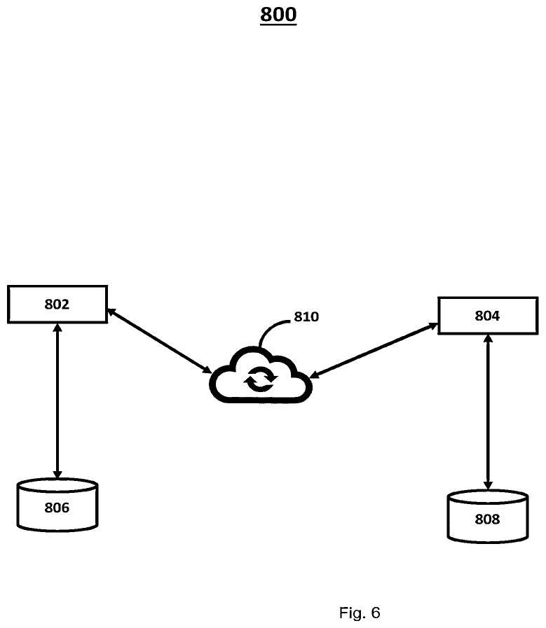

FIG. 6 is a block diagram depicting an exemplary communications architecture 800 suitable for imple-menting various embodiments as previously described.

It should be noted that the figures are purely diagrammatic and not drawn to scale. In the figures, elements which correspond to elements already described may have the same reference numerals. Examples, embodiments or optional features, whether indicated as non-limiting or not, are not to be understood as limiting the invention as claimed.

### DETAILED DESCRIPTION OF THE DRAWINGS

FIG. 1 illustrates one example of a treatment device (200) comprising one of the four different types of soil sensors: The treatment device (200) comprises a treatment arrangement (270) which is mechanically attached to the treatment device. The treatment device comprises either
a soil sensor (400/402) mechanically attached to the treatment device (200),
a soil sensor (400/404) which is not mechanically attached but communicatively coupled to the treatment device (200), and fixed on the specific location of the agricultural field, for example soil moisture sensor fixed in the soil, or
a soil sensor (400/406) which is not mechanically attached but communicatively coupled to the treatment device (200), and mobile or movable depending on the movement of the treatment device, for example a soil sensor which is part of or mounted on an aerial vehicle, an unmanned aerial vehicle, a drone, or a robot,
a soil sensor (400/408) which is not mechanically attached but communicatively coupled to the treatment device (200), and mobile or movable independent from on the movement of the treatment device, for example a soil sensor is part of or mounted on an aerial vehicle, an unmanned aerial vehicle, or a satellite

FIG. 2 illustrates one example of an executed treatment of the treatment device comprising a mechanically attached soil sensor.

The upper part of FIG. 2 illustrates the following processes: The processing unit (500) which can be either mechanically attached to the soil sensor (400/402), mechanically attached to the treatment device (200) or is located on another computing resource communicatively coupled to the soil sensor (400/402), receives real-time soil information (20)—e.g. soil reflectance data—on the real-world situation of the geographical location G1 in the agricultural field from the soil sensor (400/402) and processes this information to obtain processed information (30), e.g. the nitrogen content of the soil, which is then provided to the treatment control unit (210). The parametrization interface (240), which receives the parametrization (10) from the field manager system (100), provides the parametrization (10) to the treatment control unit (210). The treatment control unit (210) is mechanically attached or communicatively coupled to the treatment device (200), or is mechanically attached or communicatively coupled to the treatment arrangement (270). After the parametrization (10) and the processed information (30) have been provided to the treatment control unit (210), the treatment control unit (210) determines a control signal (50) for controlling the treatment arrangement (270). This control signal (50) is provided to the treatment arrangement (270).

The middle part of FIG. 2 illustrates that, during the time of the processes described in the upper part of FIG. 2, the treatment device (200) has now moved further in the agricultural field, and the treatment arrangement (270) now executes the treatment dependent on the control signal (50) on the geographical location G2 in the agricultural field.

The lower part of FIG. 2 illustrates that the distance between the geographical locations G1 and G2 does not exceed 100 meters.

FIG. 3 illustrates one example of an executed treatment of the treatment device comprising a mobile soil sensor not mechanically attached but communicatively coupled to the treatment device and moving depending on the movement of the treatment device.

The upper part of FIG. 3 illustrates the following processes: The processing unit (500) which can be either mechanically attached to the soil sensor (400/404), mechanically attached to the treatment device (200) or is located on another computing resource communicatively coupled to the soil sensor (400/404), receives real-time soil information (20)—e.g. soil reflectance data—on the real-world situation of the geographical location G1 in the agricultural field from the soil sensor (400/404) and processes this information to obtain processed information (30), e.g. the nitrogen content of the soil, which is then provided to the treatment control unit (210). The parametrization interface (240), which receives the parametrization (10) from the field manager system (100), provides the parametrization (10) to the treatment control unit (210). The treatment control unit (210) is mechanically attached or communicatively coupled to the treatment device (200), or is mechanically attached or communicatively coupled to the treatment arrangement (270). After the parametrization (10) and the processed information (30) have been provided to the treatment control unit (210), the treatment control unit (210) determines a control signal (50) for controlling the treatment arrangement (270). This control signal (50) is provided to the treatment arrangement (270).

The middle part of FIG. 3 illustrates that, during the time of the processes described in the upper part of FIG. 3, the treatment device (200) has now moved further in the agricultural field, and the treatment arrangement (270) now executes the treatment dependent on the control signal (50) on the geographical location G2 in the agricultural field.

The lower part of FIG. 3 illustrates that the distance between the geographical locations G1 and G2 does not exceed 100 meters.

FIG. 4 illustrates a flow diagram showing the operation of the method of the present invention. In step (S10), a parametrization (10) for controlling a treatment device (200) by the treatment device (200) from a field manager system (100) is received. In step (S20), real-time soil information on the real-world situation of the geographical location G1 in the agricultural field is received from a soil sensor. In step (S30), real-time soil information (20)—e.g. soil reflectance data—on the real-world situation of the geographical location G1 in the agricultural field is received from the soil sensor. In step (S40), this information is processed to obtain processed information (30), e.g. the nitrogen content of the soil. In step (S50), a treatment on the geographical location G2 in the agricultural field (300) is executed, for example by applying a treatment product to the agricultural field in a specific dosage.

The above-described methods may be embodied as instructions on a computer readable medium or as part of a computing architecture, particularly part of the computing architecture 700 as illustrated in FIG. 5. The soil sensor 400, the processing unit 500, the field manager system 100, the treatment device 200, the treatment control unit 210, the online field data interface 240, the parametrization interface 250, or the treatment arrangement 270 may be embodied as part of a computing architecture, particularly part of the computing architecture 700 as illustrated in FIG. 5.

FIG. 5 illustrates an embodiment of an exemplary computing architecture 700 suitable for implementing various embodiments as previously described. In one embodiment, the computing architecture 700 may comprise or be implemented as part of an electronic device, such as a computer 701. The embodiments are not limited in this context.

As used in this application, the terms “system” and “component” are intended to refer to a computer-related entity, either hardware, a combination of hardware and software, software, or software in execution, examples of which are provided by the exemplary computing architecture 700. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 700 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 700.

As shown in FIG. 5, the computing architecture 700 comprises a computer processing unit 702, a system memory 704 and a system bus 706. The computer processing unit 702 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi processor architectures may also be employed as the computer processing unit 702.

The system bus 706 provides an interface for system components including, but not limited to, the system memory 704 to the computer processing unit 702. The system bus 706 can be any of several types of bus structure that may further interconnect to a memory bus (with or without a memory controller), a peripheral bus, and a local bus using any of a variety of commercially available bus architectures. Interface adapters may connect to the system bus 706 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The computing architecture 700 may comprise or implement various articles of manufacture. An article of manufacture may comprise a computer-readable storage medium to store logic.

Examples of a computer-readable storage medium may include any tangible media capable of storing electronic data, including volatile memory or non-volatile memory, removable or non-removable memory, erasable or non-erasable memory, writeable or re-writeable memory, and so forth. Examples of logic may include executable computer program instructions implemented using any suitable type of code, such as source code, compiled code, interpreted code, executable code, static code, dynamic code, object-oriented code, visual code, and the like. Embodiments may also be at least partly implemented as instructions contained in or on a non-transitory computer-readable medium, which may be read and executed by one or more processors to enable performance of the operations described herein.

The system memory 704 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 5, the system memory 704 can include non-volatile memory 708 and/or volatile memory 710. A basic input/output system (BIOS) can be stored in the non-volatile memory 708.

The computing architecture 700 may include various types of computer-readable storage media in the form of one or more lower speed memory units, including an internal (or external) hard disk drive (HDD) 712, a magnetic floppy disk drive (FDD) 714 to read from or write to a removable magnetic disk 716, and an optical disk drive 718 to read from or write to a removable optical disk 720 (e.g., a CD-ROM or DVD). The HDD 712, FDD 714 and optical disk 720 can be connected to the system bus 706 by an HDD interface 722, an FDD interface 724 and an optical drive interface 726, respectively. The HDD interface 722 for external drive implementations can include at least one or both of Universal Serial Bus (USB) and IEEE 694 interface technologies.

The drives and associated computer-readable media provide volatile and/or nonvolatile storage of data, data structures, computer-executable instructions, and so forth. For example, a number of program modules can be stored in the drives and memory units 708, 712, including an operating system 728, one or more application programs 730, other program modules 732, and program data 734. In one embodiment, the one or more application programs 730, other program modules 732, and program data 734 can include, for example, the various applications and/or components of the #the soil sensor 400, processing unit 500, the field manager system 100, the treatment device 200, the treatment control unit 210, the online field data interface 240, the parametrization interface 250, or the treatment arrangement 270.

A user can enter commands and information into the computer 701 through one or more wire/wireless input devices, for example, a keyboard 736 and a pointing device, such as a mouse 738. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the computer processing unit 702 through an input device interface 740 that is coupled to the system bus 706, but can be connected by other interfaces such as a parallel port, IEEE 694 serial port, a game port, a USB port, an IR interface, and so forth.

A monitor 742 or other type of display device is also connected to the system bus 706 via an interface, such as a video adaptor. The monitor 742 may be internal or external to the computer 701. In addition to the monitor 742, a computer typically includes other peripheral output devices, such as speakers, printers, and so forth.

The computer 701 may operate in a networked environment using logical connections via wire and/or wireless communications to one or more remote computers, such as a remote computer 744. The remote computer 744 can be a workstation, a server computer, a router, a personal computer, portable computer, microprocessor-based entertainment appliance, a peer device or other common network node, and typically includes many or all of the elements described relative to the computer 701, although, for purposes of brevity, only a memory/storage device 746 is illustrated. The logical connections depicted include wire/wireless connectivity to a local area network (LAN) 748 and/or larger networks, for example, a wide area network (WAN) 750. Such LAN and WAN networking environments are commonplace in offices and companies, and facilitate enterprise-wide computer networks, such as intranets, all of which may connect to a global communications network, for example, the Internet.

When used in a LAN networking environment, the computer 701 is connected to the LAN 748 through a wire and/or wireless communication network interface or adaptor 752. The adaptor 752 can facilitate wire and/or wireless communications to the LAN 748, which may also include a wireless access point disposed thereon for communicating with the wireless functionality of the adaptor 752.

When used in a WAN networking environment, the computer 701 can include a modem 754, or is connected to a communications server on the WAN 750, or has other means for establishing communications over the WAN 750, such as by way of the Internet. The modem 754, which can be internal or external and a wire and/or wireless device, connects to the system bus 706 via the input device interface 740. In a networked environment, program modules depicted relative to the computer 701, or portions thereof, can be stored in the remote memory/storage device 746. It will be appreciated that the network connections shown are exemplary and other means of establishing a communications link between the computers can be used.

The computer 701 is operable to communicate with wire and wireless devices or entities using the IEEE 802 family of standards, such as wireless devices operatively disposed in wireless communication (e.g., IEEE 802.13 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.13x (a, b, g, n, etc.) to provide secure, reliable, fast wireless connectivity. A Wi-Fi network can be used to connect computers to each other, to the Internet, and to wire networks (which use IEEE 802.3-related media and functions).

FIG. 6 is a block diagram depicting an exemplary communications architecture 800 suitable for implementing various embodiments as previously described. The communications architecture 800 includes various common communications elements, such as a transmitter, receiver, transceiver, radio, network interface, baseband processor, antenna, amplifiers, filters, power supplies, and so forth. The embodiments, however, are not limited to implementation by the communications architecture 800.

As shown in FIG. 6, the communications architecture 800 includes one or more clients 802 and servers 804. The clients 802 and the servers 804 are operatively connected to one or more respective client data stores 806 and server data stores 808 that can be employed to store information local to the respective clients 802 and servers 804, such as cookies and/or associated contextual information.

The clients 802 and the servers 804 may communicate information between each other using a communication framework 810. The communications framework 810 may implement any wellknown communications techniques and protocols. The communications framework 810 may be implemented as a packet-switched network (e.g., public networks such as the Internet, private networks such as an enterprise intranet, and so forth), a circuit-switched network (e.g., the public switched telephone network), or a combination of a packet-switched network and a circuit-switched network (with suitable gateways and translators).

The communications framework 810 may implement various network interfaces arranged to accept, communicate, and connect to a communications network. A network interface may be regarded as a specialized form of an input output interface. Network interfaces may employ connection protocols including without limitation direct connect, Ethernet (e.g., thick, thin, twisted pair 10/100/1000 Base T, and the like), token ring, wireless network interfaces, cellular network interfaces, IEEE 802.8a-x network interfaces, IEEE 802.16 network interfaces, IEEE 802.20 network interfaces, and the like. Further, multiple network interfaces may be used to engage with various communications network types. For example, multiple network interfaces may be employed to allow for the communication over broadcast, multicast, and unicast networks. Should processing requirements dictate a greater amount speed and capacity, distributed network controller architectures may similarly be employed to pool, load balance, and otherwise increase the communicative bandwidth required by clients 802 and the servers 804. A communications network may be any one and the combination of wired and/or wireless networks including without limitation a direct interconnection, a secured custom connection, a private network (e.g., an enterprise intranet), a public network (e.g., the Internet), a Personal Area Network (PAN), a Local Area Network (LAN), a Metropolitan Area Network (MAN), an Operating Missions as Nodes on the Internet (OMNI), a Wide Area Network (WAN), a wireless network, a cellular network, and other communications networks.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the methods or computer-implemented methods described herein.

1. A method for treatment of an agricultural field (300), the method comprising the steps:
1) receiving (S10) a parametrization (10) for controlling a treatment device (200) by the treatment device (200) from a field manager system (100);
2) receiving (S20)—from at least one soil sensor (400)—real-time soil information on the real-world situation of the geographical location G1 in the agricultural field (300);
3) processing (S30) the real-time soil information to generate processed information (30);
4) determining (S40) a control signal (50) for controlling a treatment arrangement (270) of the treatment device (200) based on the received parametrization (10) and the processed information (30); and
5) executing (S50) a treatment on the geographical location G2 in the agricultural field (300),
wherein the treatment is executed based on the control signal (50) real-time after receiving the real-time soil information in such a way that the distance between location G1 and location G2 does not exceed 100 meters.

2. A method according to claim 1, wherein the parametrization (10) is dependent on offline field data (Doff) relating to expected conditions on the agricultural field (300).

3. A method according to claim 1, comprising the additional steps:
receiving the offline field data (Doff) by the field manager system (100);
determining the parametrization (10) of the treatment device (200) dependent on the offline field data (Doff) and determining a dosage level (40) or determining at least one treatment product type (41); and
providing the determined parametrization (10) and the determined dosage level (40) or the determined treatment product type (41) to the treatment device (200).

4. A method according to claim 1, wherein the physical distance between the soil sensor (400) and the soil is less than 100 cm at the time of obtaining real-time soil information on the real-world situation in the agricultural field (300).

5. A method according to claim 1, wherein the soil sensor (400) is a non-optical spectrometer, an optical spectrometer, an infrared spectrometer, an electric conductivity sensor, a magnetic susceptibility (EM) sensor, a gamma-ray sensor, a Lidar sensor, a near-infrared sensor, or a photoconductive-layer-containing optical sensor.

6. A method according to claim 1, wherein the soil sensor (400) is an infrared spectrometer optionally supplemented by one of the sensors selected from non-optical spectrometer, optical spectrometer, electric conductivity sensor, gamma-ray sensor, magnetic susceptibility (EM) sensor, and/or optionally supplemented by a camera.

7. A method according to claim 1, wherein the soil sensor (400) is a photoconductive-layer-containing optical sensor optionally supplemented by one of the sensors selected from non-optical spectrometer, optical spectrometer, electric conductivity sensor, gamma-ray sensor, magnetic susceptibility (EM) sensor, and/or optionally supplemented by a camera.

8. A method according to claim 1, wherein the soil sensor (400) is mechanically attached to the treatment device (200).

9. A method according to claim 1, wherein the soil sensor (400) is not mechanically attached to the treatment device (200) and is directly or indirectly communicatively coupled to the treatment device (200).

10. A method according to claim 1, wherein the treatment device (200) is designed as a smart seed applicator, wherein the treatment arrangement (270) is a seeding arrangement.

11. A method according to claim 1, wherein the treatment device (200) is designed as a smart fertilizer applicator, wherein the treatment arrangement (270) is a fertilizing arrangement.

12. A method according to claim 1, wherein the treatment device (200) is designed as a smart sprayer, wherein the treatment arrangement (270) is a nozzle arrangement.

13. A method according to claim 1, wherein the treatment device (200) is designed as a smart irrigation applicator, wherein the treatment arrangement (270) is an irrigation arrangement.

14. A method according to claim 1, comprising the steps:
receiving online field data (Don) by the treatment device (200) relating to current conditions on the agricultural field (300); and
determining the control signal (50) dependent on the determined parametrization (10), the processed information (30), and the determined online field data (Don).

15. A method according to claim 14, wherein the online field data (Don) relates to current machine data, weather condition data, and current plantation growth data.

16. A method according to claim 1, comprising the step:
adjusting the parametrization (10) and/or the dosage level (40) or the at least one treatment product type (41) using a machine learning algorithm.

17. A method according to claim 1, comprising the step:
processing (S30) the real-time soil information to generate processed information (30) using a machine learning algorithm.

18. A method according to claim 1, wherein
determining a parametrization (10) comprises determining a tank recipe for a treatment product tank of the treatment device (200).

19. A treatment device (200) for treatment of an agricultural field (300), comprising:
a soil sensor (400);
a processing unit (500) being adapted for processing the real-time soil information on the real-world situation of the geographical location G1 in the agricultural field (300) as received from the soil sensor (400) and generating processed information (30);
a parametrization interface (250) being adapted for receiving a parametrization (10) from a field manager system (100);
a treatment arrangement (270) being adapted for treating the agricultural field (300) dependent on the control signal (50) and being adapted for executing a treatment on the geographical location G2 in the agricultural field (300) real-time after receiving the real-time soil information in such a way that the distance between location G1 and location G2 does not exceed 100 meters; and
a treatment control unit (210) being adapted for determining a control signal (50) for controlling a treatment arrangement (270) based on the parametrization (10) which it receives from the parametrization interface (240) and based on the processed information (30).

20. The treatment device of claim 19, comprising
an online field data interface (240) being adapted for receiving online field data (Don) relating to current conditions on the agricultural field (300), wherein
the treatment control unit (210) is adapted for determining a control signal (50) for controlling a treatment arrangement (270) dependent on the received parametrization (10) and the processed information (30) and/or the online field data (Don).
